(19) United States
(12) Patent Application Publication
(10) Pub. No.: US 2024/0157035 A1
(43) Pub. Date: May 16, 2024

(54) FLUID SPLITTING INTO FLUID PRODUCTS WITH DESIGNATED CHARACTERISTICS

(71) Applicant: Fenwal, Inc., Lake Zurich, IL (US)

(72) Inventors: James Madsen, Chicago, IL (US); Kyungyoon Min, Kildeer, IL (US)

(21) Appl. No.: 18/504,088

(22) Filed: Nov. 7, 2023

Related U.S. Application Data
(60) Provisional application No. 63/382,763, filed on Nov. 8, 2022.

(51) Int. Cl. A61M 1/02

(52) U.S. Cl. CPC A61M 1/0281; A61M 1/0209; A61M 2202/0427; A61M 2205/10; A61M 2205/123; A61M 2205/3327; A61M 2205/3393; A61M 2205/50; A61M 2209/08

(57) ABSTRACT

A system and method for splitting a fluid into fluid products with a component count within a designated component count range and volume within a designated volume range. The method includes measuring an initial component concentration of a source container, weighing a source container to determine the volume of fluid, calculating the final component count and final volume for each of the source and at least one satellite container, calculating the amount of component additive solution to add the source container and flowing the amount of component additive solution from a component additive solution container into the source container, and splitting the fluid so that each divided fluid amount has a final component count within a designated component count range and a final volume within a designated volume range.

### CROSS-REFERENCE TO RELATED APPLICATIONS

This application claims the benefit of and priority of U.S. Provisional Patent Application Ser. No. 63/382,763, filed Nov. 8, 2022, the contents of which are incorporated by reference herein.

### FIELD OF THE DISCLOSURE

The present disclosure relates to automated splitting of a fluid. More particularly, the present disclosure relates to apparatus and methods for splitting a fluid into two or more fluid products resulting in at least two products having a fluid component count and fluid volume within designated ranges.

### DESCRIPTION OF RELATED ART

It is well known to separate blood into its constituents, including separating and collecting a platelet or other component product. A single apheresis platelet donation unit typically contains approximately 3.0×10¹¹ platelets suspended in a volume of approximately 200-400 ml of plasma, although a single apheresis procedure may produce a platelet product having a much greater volume, such as approximately 9.6×10¹¹ platelets suspended in a volume of approximately 700 ml of plasma. In such a scenario, it is typical to split the high-volume platelet product into separate amounts each having a volume within the range of what is acceptable for a single unit, with a high-volume platelet product typically being split into either two or three units having approximately the same volumes.

The high-volume platelet product is conventionally split into individual units according to a manual approach. However, more recently, an automated approach may be utilized, as described in US Patent Publication No. 2022/0260408, the disclosure of which is incorporated by reference in its entirely.

Before using platelet products in procedures or treatments, the products are often treated and/or tested in order to reduce infection risk. Pathogen Reduction Treatment (PRT) is often a preferred method to reduce infection risk. PRT requires a specific platelet count range and volume range for treatment, called guard bands. If a platelet product does not meet these platelet count and volume requirements it cannot be used in PRT and may have to undergo lengthy bacterial testing during which a sample is held in culture for 24-36 hours to check for bacterial contamination.

An automatic splitting method and device which evaluates a platelet product and determines an amount of (platelet) additive solution to be added to the product before splitting into multiple products which meet the count and volume requirements for PRT or another type of treatment is therefore desirable.

### SUMMARY

There are several aspects of the present subject matter which may be embodied separately or together in the devices, systems, and methods described and/or claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto or later amended.

In one aspect, a method of splitting a fluid into fluid products with a component count within a designated component count range and a volume within a designated range includes establishing a designated component count range and designated volume range for fluids in each of a source container and at least one satellite container, weighing a source container to determine the volume of the fluid, and inputting the initial component concentration and initial volume into the splitting device. The method further includes calculating the final component count and final volume for the fluid of each of the source and at least one satellite container, comparing the final component count to the designated component count range and the final volume to the designated volume range for each container, calculating the amount of component additive solution to add to the source container so that each of the source and satellite container have a fluid with a final component count and final volume within the designated component count range and volume range, and flowing the amount of component additive solution from a component additive solution container into the source container. Steps are repeated until there is no amount of additive solution needed and the calculated final component count and final volume for the fluid of each of the source container and at least one satellite container is within the designated component count range and the final volume is within the designated volume range. The method further includes flowing fluid from the source container of a fluid flow circuit to at least one satellite container of the fluid flow circuit by selectively allowing and preventing fluid flow from the source container to the at least one satellite container resulting in a satellite container and source container containing fluid with a final component count within a designated component count range and a final volume within a designated volume range.

In yet another aspect, a system for splitting a fluid includes a source support configured to support a source container of a fluid flow circuit and at least one satellite support configured to support at least one satellite container of the fluid flow circuit fluidly connected to the source container. The system also includes a weight scale associated with each of the supports, at least one clamp, and an input. The system further includes a controller configured to establish a designated component count range and designated volume range for fluid in each of a source container and at least one satellite container, measure an initial component count of the fluid in a source container, weigh a source container to determine the volume of fluid and input an initial component concentration and initial volume into the splitting device. The controller is also configured to calculate the final component count and final volume for fluid in each of the source and at least one satellite container, compare the final component count to the designated component count range and the final volume to the designated volume range for each container, calculate the amount of component additive solution to add to the source container so that each of the source and at least one satellite container have a fluid with a final component count and final volume within the designated component count range and volume range and flow the amount of component additive solution from a component additive solution container into the source container. The controller repeats these steps until there is no component additive solution amount needed and the calculated final component count for each of the fluid in the source and at least one satellite container is within the designated count range and the final volume is within the designated volume range. The controller is further configured to flow fluid from the source container of a fluid flow circuit to at least one satellite container of the fluid flow circuit by selectively allowing and preventing fluid flow from the source container to the at least one satellite container resulting in a satellite container and source container with a final component count within a designated component count range and a final volume within a designated volume range.

### BRIEF DESCRIPTION OF THE DRAWINGS

FIG. 1 is a front elevational view of an exemplary embodiment of a system for supporting a fluid container and splitting the fluid into at least two products, according to an aspect of the present disclosure.

FIG. 2A is a front elevational view of a first step of a method of splitting a fluid according to an aspect of the present disclosure.

FIG. 2B is a front elevational view of an alternate first step of a method of splitting a fluid according to an aspect of the present disclosure.

FIG. 3A is a front elevational view of a second step of a method of splitting a fluid according to an aspect of the present disclosure.

FIG. 3B is a front elevational view of an alternate second step of a method of splitting a fluid according to an aspect of the present disclosure.

FIG. 4 is a front elevational view of a third step of a method of splitting a fluid according to an aspect of the present disclosure.

FIG. 5 is a front elevational view of a fourth step of a method of splitting a fluid according to an aspect of the present disclosure.

### DETAILED DESCRIPTION OF THE ILLUSTRATED EMBODIMENTS

The embodiments disclosed herein are for the purpose of providing an exemplary description of the present subject matter. They are, however, only exemplary and not exclusive, and the present subject matter may be embodied in various forms. Therefore, specific details disclosed herein are not to be interpreted as limiting the subject matter as defined in the accompanying claims.

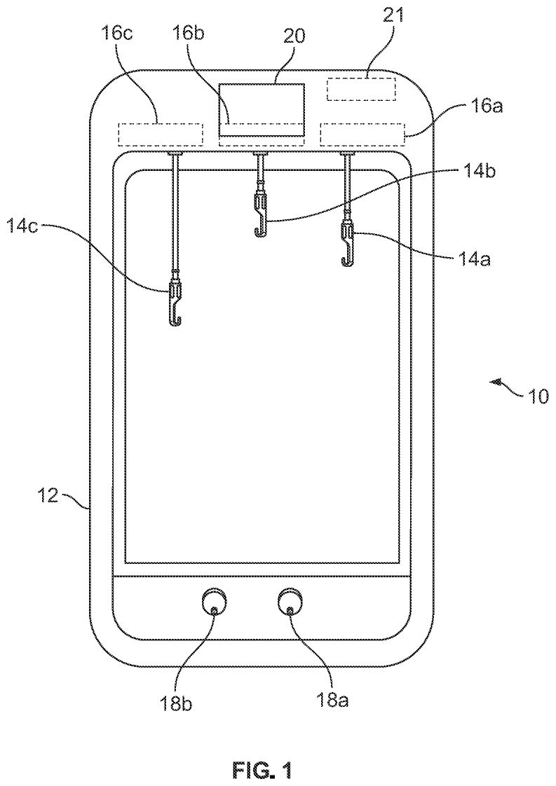

FIG. 1 depicts an exemplary system 10 for splitting a fluid into two or more amounts. A system 10 of the type shown in FIG. 1 may be particularly advantageous for splitting a platelet product or other component blood product into two or more amounts, but it should be understood that the techniques described herein are not limited to the splitting of platelet products, but may be applied to other biological fluids and other fluid components. For example, the component can be any component desirable for a particular treatment, including, but not limited to, components such as platelets, red or white blood cells, T-cells, stems cells, etc. Typically, these cells are separated from a base starting fluid such as whole blood, with a unit or other amount of whole blood being processed to produce a platelet product having some amount of the component. Any of a number of possible approaches may be applied to separate the components from the base fluid, including (but not limited to) centrifugation, separation via a spinning membrane, and filtration. Specifically, the blood may be processed by apheresis and resulting fluid is an apheresis product, which is split in the below discussed procedure.

FIG. 1 shows such a system 10 for separating a fluid into separate fluid products, each fluid product meeting designated characteristics such as platelet count range and measured volume range. More particularly, the system 10 includes a frame 12, at least one container support member 14 (individually identified in FIG. 1 as 14a, 14b, and 14c) configured to engage and support a fluid container, a user input 20, a controller 21, clamps 18 (individually 18a and 18b) and at least one weight scale 16 (individually identified in FIG. 1 as 16a, 16b, and 16c). The system 10 may include additional components without departing from the scope of the present disclosure, such as pumps associated with the system 10.

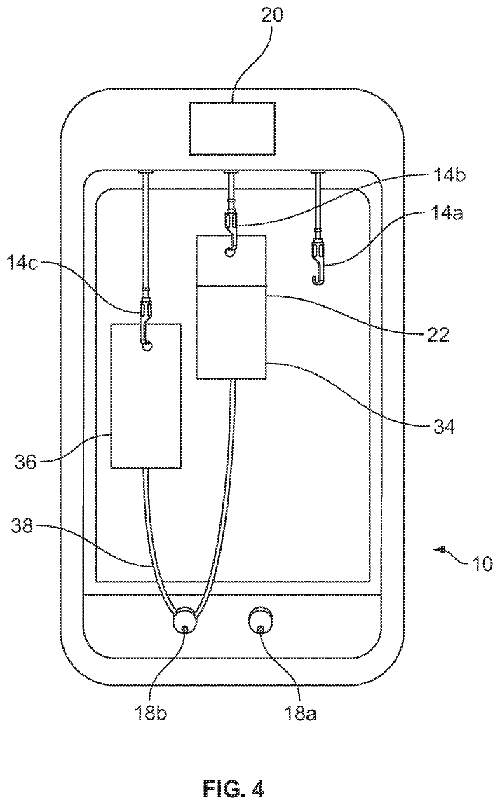

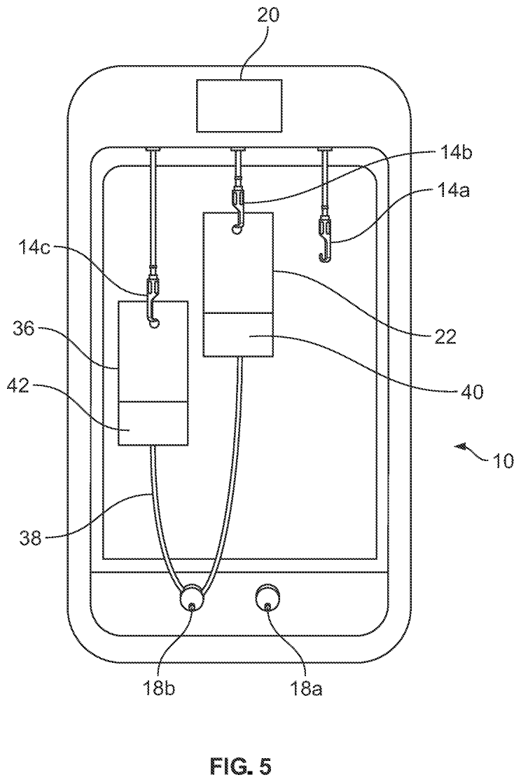

The illustrated system 10 includes a frame 12 (at least partially formed of a metallic or rigid material in one embodiment) having a container support 14 configured to support containers of a fluid flow circuit. The fluid flow circuit, when attached, may include a source container 22 and at least one satellite container 36 (shown in FIGS. 4 and 5). The system 10 is configured as a durable, reusable device, while the fluid flow circuit, which may be attached to the system 10, is typically disposable and configured as a single-use item. However, it is within the scope of the present disclosure for the fluid flow circuit to be configured as a reusable item.

The particular configuration of the container supports 14 may vary without departing from the scope of the present disclosure and may depend upon the nature of the container that it is intended to support (with the configuration of the container also being subject to variation without departing from the scope of the present disclosure). For example, in one embodiment, the containers are configured as flexible bags having an upper opening or aperture. In this case, the container support 14 may include or be configured as a hook or hanger, as shown in FIG. 1, which includes a portion that extends into and through the upper opening or aperture of the container to support and suspend the container at some elevation. FIG. 1 illustrates the container supports as hooks, but the container support members may be differently configured, such as a ring, a clip or clamp, with each being appropriate for use in combination with a differently configured fluid container. It should be understood that other configurations may also be employed without departing from the scope of the present disclosure. Although all of the container supports 14 are shown similarly configured, they may be differently configured and dependent on the specific container they are supporting. The container supports can be designated as source and satellite container supports, with support 14b being used as the source container support and supports 14a and 14c used for satellite containers. However, it is within the scope of this disclosure for source and satellite container(s) to be associated with any of the container supports 14.

The system 10 also includes at least one clamp 18 (individually labeled as 18a and 18b in the FIGS.). The containers are fluidly connected to each other by a conduits (e.g., a tube), with the conduits being placed into association with the clamps when a fluid flow circuit is attached to the frame 12. The clamps, which may be variously configured without departing from the scope of the present disclosure, are configured to selectively allow and prevent fluid flow between the containers (namely, the source and satellite container). In one embodiment, the clamps may be configured to be automatically (i.e., non-manually) moved between closed and open conditions. In the open condition, the clamp or valve allows fluid flow through the conduit. In the closed condition, the clamp or valve prevents fluid flow through the conduit. The manner in which the clamp or valve prevents fluid flow through the conduit may vary depending on the configurations of the clamp or valve and the conduits. For example, in one embodiment, the conduit is configured as a flexible tube, with the clamp or valve configured as a pinch valve, which may squeeze the conduit to close it, thereby preventing fluid flow through the conduit. If the conduit is differently configured (e.g., as a rigid tube), the clamps may be differently configured (e.g., as a ball valve) to selectively allow and prevent fluid flow through the conduit. Regardless of the particular configuration of the clamp, the operation of the clamp is automatic (rather than manual), being controlled by the controller of the system. This may include the clamp being controlled in accordance with the fluid splitting procedure described below.

The system 10 may include at least one position adjustment assembly (not shown). There may be one position adjustment assembly associated with each container support, but there may be more or less depending on the container supports and requirement for adjustment. Each position adjustment assembly is configured to allow for adjustment of a position of the container support member with respect to the frame 12.

There are many different configurations for a position adjustment assembly. By way of example, the position adjustment assembly may employ a mechanically actuatable adjustment assembly such as a screw or threaded rod, a wheel and an axle, or a cam and a wheel. The adjustment assembly may also be electromechanically actuatable and employ an electro-mechanical screw actuator with an associated motor, a wheel and a motor, or a cam, connection member and a retaining spring. The assembly may utilize electrical, pneumatic, or hydraulic fluid pressure energy.

The adjustment assembly may also include a cylinder. In such an embodiment, the cylinder may be hollow and contain a piston that is configured to move through the cylinder. Such an adjustment assembly also includes a fluid supply reservoir that fluidically communicates with the cylinder. The adjustment assembly may alternatively (or additionally) include a magnetic linear motor. In another embodiment, the adjustment assembly may include a telescoping member. The adjustment assembly may include a piezoelectric member, which is at least partially comprised of one or more materials designed to expand or contract with the application of electrical voltage. The adjustment assembly may also include a shape memory alloy.

Each support 14 may have a weight scale 16 associated with it. The weight scale may be a component of the position adjustment assembly or a separate component of the system 10. The weight scales may be similarly or differently configured. The weight scales may work independently or function in a combined manner. Regardless of the particular configuration, each weight scale is configured to measure a combined weight of the container that it is supporting (i.e., the tare weight) and the contents of that container. By knowing the weight of the empty container and the combined weight, the weight of any fluid in the container may be calculated by subtracting the tare weight from the combined weight. The volume can be determined from the measured weight.

The weight scale 16 of an associated container is configured to transmit signals to the system controller that are indicative of the combined weight of the supported container (i.e., the tare weight) and its contents, with the controller using such information to execute a fluid splitting procedure, as described in greater detail below. The weight scale of the container support may be variously configured without departing from the scope of the present disclosure, with the weight scale including a load cell in one embodiment.

System 10 may also include a user input 20. The input 20 may include a number of different devices according to the embodiments described herein. The input provides a mode of communication from the operator to the device and the device to the operator. The input 20 may be a touch screen. Alternatively, or in addition, the input 20 could include a keyboard or keypad. In each instance, a user may provide information and/or instructions to the controller 21. These instructions may include type of procedure, measurements of fluid containers, and when to begin/stop a procedure. The input could also include a reader or scanner, such as a barcode reader or scanner or an RFID reader.

The input is coupled to the controller 21. The controller 21 receives information from the input and may also communicate information or commands to the input 20. According to still other embodiments, the input 20 may be in the form of computer equipment that permits the splitting device including the controller 21 to communicate (whether via wires, cables, etc. or wirelessly) with other systems over a local network, or with other cell processing systems or other computer equipment (e.g., a server) over local networks, wide area networks, or the Internet. According to such an embodiment, the input may include an internal transmitter/receiver device.

The system may also include at least one indicator associated with the controller. The indicator may be incorporated anywhere into the system without departing from the scope of the present disclosure, it may be part of the input or a separate component. The indicator is configured to display at least one of an indication of a status of the system, which may include (for example) when the system is ready to begin processing, when the system is processing, when the system has completed processing, and when there has been an error. The indicator may display or represent the status of the fluid-splitting system in any suitable manner.

The system 10 can also include buttons or icons associated with the controller. The buttons or icons may be variously configured and positioned at any suitable location of the system. One button/icon may be a start button/icon for initializing a splitting procedure and the other button/icon of the set may be a stop button/icon for stopping a splitting procedure. These buttons may be integrated within the input or separate for performing separate functions.

The weight scales, input, and the clamps communicate with controller 21. The controller 21 carries out process control and monitoring functions for the system 10. The controller 21 comprises a main processing unit (MPU), which can comprise, e.g., a PENTIUM® type microprocessor made by Intel Corporation, although other types of conventional microprocessors can be used. In the illustrated embodiment, the controller 21 is incorporated into the frame 12, but it should be understood that the controller 21 may be incorporated into a separate component of the system 10, such as a computer that is associated with the weight scales and the clamps 18 by a wired or wireless connection.

According to other embodiments, the controller 21 may include one or more electrical circuits designed to carry out the actions described herein. In addition, the controller 21 may include one or more memories. The instructions by which the microprocessor is programmed may be stored on the memory associated with the microprocessor, which memory/memories may include one or more tangible non-transitory computer readable memories, having computer executable instructions stored thereon, which when executed by the microprocessor, may cause the microprocessors to carry out one or more actions as described below.

The controller 21 may be coupled to one or more of the structures described above, for example to receive information (e.g., in the form of signals) from these structures or to provide commands (e.g., in the form of signals) to these structures to control the operation of the structures. The controller 21 may be coupled to scales, the clamps and input 20 to receive information from those components. The controller 21 may be directly electrically connected to these structures to be coupled to them, or the controller 21 may be directly connected to other intermediate equipment that is directly connected to these structures to be coupled to them.

The controller is configured and/or programmed to execute at least one fluid splitting procedure but, more advantageously, is configured and/or programmed to execute a fluid splitting procedure which produces split fluids meeting specific criteria or characteristics.

More particularly, in carrying out any fluid splitting procedure, the controller is configured and/or programmed to control the flow of a fluid and amount of a fluid from one container to another (for instance, from an additive solution container to a source container or from a source container to at least one satellite container). This may include instructing the clamp or clamps to open and close at specific points or initiating a weight scale to measure and input the weight of a container. Hence, while it may be described herein that a particular component of the splitting system performs a particular function, it should be understood that that component is being controlled by the controller to perform that function.

Before, during, and after a procedure, the controller may receive readings from the weight scales or input provided by the user and may have to adjust based on these readings. For example, the controller may instruct one of the clamps to close or the input to notify a user of the status of the procedure.

Turning now to a method of splitting by the system 10, the system 10 can be used in a series of steps to ensure that the split fluids have a particular final component count and final volume in designated ranges in a container. Commonly, a collected platelet product (such as from an apheresis process) contains platelets in an amount higher than needed for a treatment or procedure. Therefore, it is common to split this product into multiple lesser fluid amounts to be utilized in a procedure or treatment. In order to be used in a procedure or treatment, a split fluid product may need to be further processed. That processing may require the fluid to have specific characteristics. For instance, the volume of fluid may need to be within a specific range. Additionally, it may be desirable that the count of platelets or another component are within a certain range.

One example of further processing is Pathogen Reduction Treatment (PRT). In PRT it is often required that the volume and platelet count be within a certain range. This range may be a required dose for treatment. Table 1 below shows example required volume ranges and platelet count ranges. However, for other procedures or treatments the split fluid may be required to have different characteristics. When a different component is of interest the range may be different. As applied to PRT testing, the platelet count range may be between 2.9×10¹¹ and 8.0×10¹¹, for small volume PRT preferably between 2.9×10¹¹ and 5.0×10¹¹, for large volume PRT preferably between 3.0×10¹¹ and 6.0×10¹¹, and for dual storage PRT preferably between 6.1×10¹¹ and 8.0×10¹¹. As applied to PRT testing, the product volume may be between 255 mL and 420 mL, for small volume PRT preferably between 255 mL and 325 mL, for large volume PRT preferably between 325 mL and 390 mL, and for dual storage PRT preferably between 375 mL and 420 mL. Table 1 also shows the limited requirements for the BacT treatment.

TABLE 1

| Platelet Count | Product Volume | |
|---|---|---|
| (×10¹¹) | (mL) | Test Label |
| ≥2.9 and ≤5.0 | ≥255 and ≤325 | PRT, Small Volume (SV) |
| ≥3.0 and ≤6.0 | ≥325 and ≤390 | PRT, Large Volume (LV) |
| ≥6.1 and ≤8.0 | ≥375 and ≤420 | PRT, Dual Storage (DS) |
| ≥3.0 | ≥200 | BacT |

System 10 may be utilized in order to ensure that the split fluid has a component count and volume within a designated range. Before initializing a split, the system may be preprogrammed with values or a user may input a value or range of values, such as shown above, for acceptable ranges of platelet count and product volume. A user may use the input 20 to set these ranges or select from list of ranges/amounts. A user may also select a procedure or treatment, such as PRT and designate the test label as shown above, with the device automatically uploading the required or designated component count range and volume range. The device/input/controller may also be connected to a remote device or computer for updating requirements for product component count and volume, as product standards and testing requirements may change for any procedure or treatment.

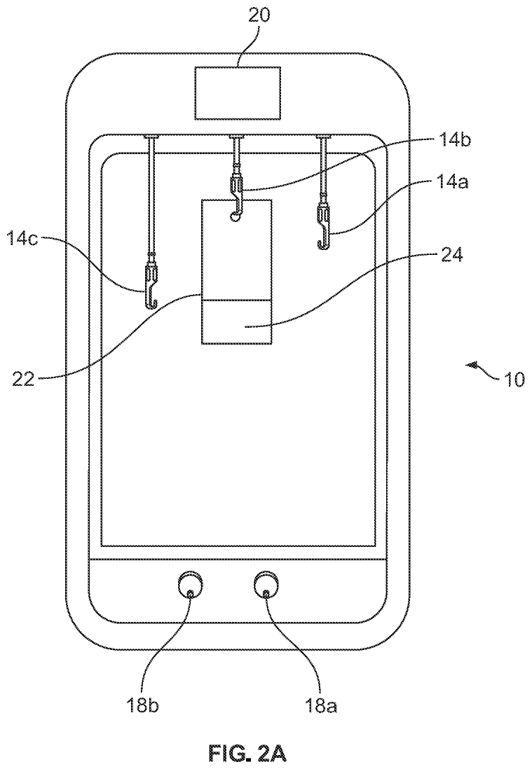

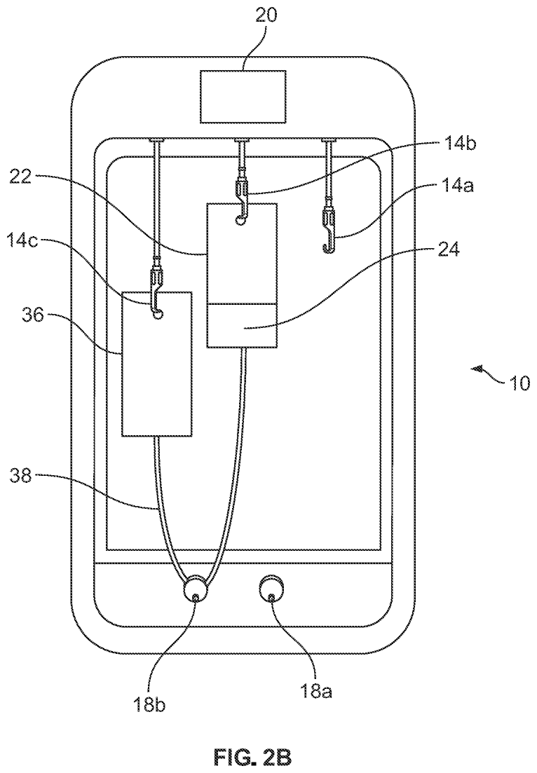

FIGS. 2A and 2B show alternate first stages in a splitting procedure. The only difference between FIGS. 2A and 2B is the connection of the source bag 22 to the satellite bag 36, with 2A displaying an embodiment wherein the source bag and satellite bag are not pre-connected and 2B displaying an embodiment wherein the source bag and the satellite bag are pre-connected. The source product bag is hung on a first support 14b. If pre-connected, the satellite bag 36 is hung on a second support 14c. The source product bag has an amount of product 24. The product bag 22 is weighed and this is used to calculate the volume of fluid in the container. The controller may use designated container weights or require an extra step to assign an empty or tare weight. The platelet concentration, which may be previously determined by another measurement, is entered into the input. The concentration of the component, such as platelets, can be measured by any known method. In an exemplary embodiment, the amount of component is measured by optical detection. In other exemplary embodiments, the amount of component is measured as part of complete blood count, by flow cytometry, or any other cell quantification method completed by an automated cell counting system. On the input, the user can then calculate the split, which is calculated by the controller by utilizing the input number and weight/calculated volume. The controller determines and displays on the input screen what the split values would be and whether they will meet the required count and volume amounts. If the split fluid values are within the designated component count and volume ranges, the system/input will prompt the user to hang at least one satellite bag so that the split can be completed.

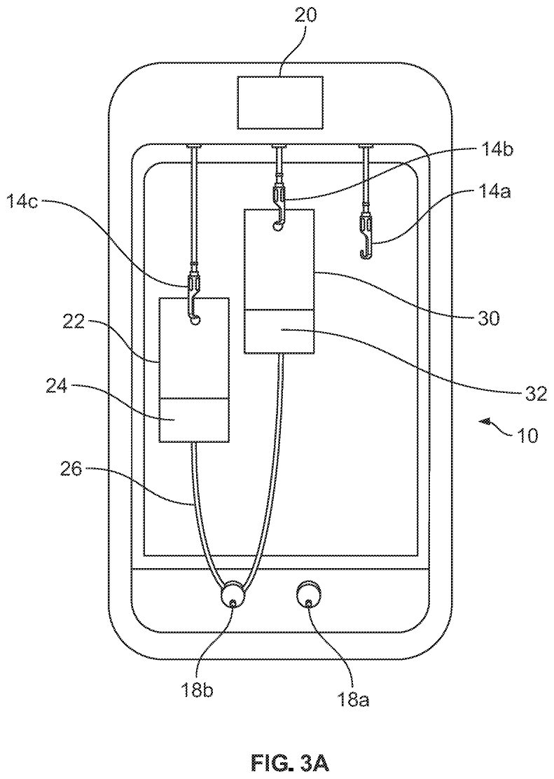

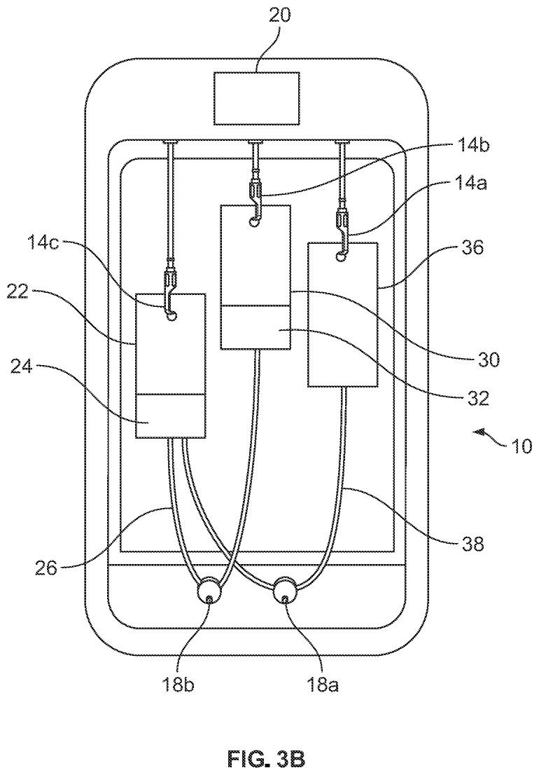

If the split fluid values will not meet the required count and volume, the user is notified. The controller will calculate the amount of an additive solution, such as platelet additive solution (PAS), that can be added to the source product container 22 in order to make a split with each split bag having a final component count and final fluid volume in the designated range. This prompts the user to remove the source product bag from the middle container support 14b and move the source product bag to a second container support. FIG. 3A shows the source product bag as being moved to container support 14c, but a user may choose instead to move the source product bag to container support 14a. In the embodiment of FIG. 2B, where the source and satellite bag are pre-connected, the satellite bag is moved to a different support, such as 14a.

Once the product source bag 22 is moved, the user can attach a PAS product bag with an amount of additive solution, or PAS 32, to container support 14b and sterilely connect the PAS product bag 30 to the source product bag 22 via a tube or conduit 26, through the clamp 18b. Once connected, the user can initiate the transfer of the calculated volume of PAS from the PAS product bag 30 to the source product bag 22, increasing the amount of fluid 24 in the source product bag by making some type of action or input or the weight scales may sense the bags are present and the controller may initiate moving to the next step.

Once the product source bag 22 is filled with a new amount of fluid to be split 34, the controller stops the transfer, and the user can disconnect and remove the PAS bag and hang the source product bag 22, with the new amount of fluid 34 in the source container 22 on the center container support. The user then inputs the adjusted amount of component concentration after a measurement is done or the system automatically is updated by the controller to the new platelet concentration and the bag is weighed for fluid amount. On the input, the user can initiate a split calculation, which is calculated by the controller by utilizing the input number and weight. The controller determines and displays on the input display what the split values would be and whether they will fall within the required or desired count and volume ranges. If the split fluid values meet the component count and volume amounts, the system/input will prompt the user to hang and sterilely connect at least one satellite bag so that the split can be completed (unless with the alternate steps 2B and 3B wherein the satellite bag is already connected to the source bag). As shown in FIG. 4, the user can then sterilely attach a satellite product bag 36 and fluidly connect the source product bag 22 and the satellite product bag through conduit 38 and clamp 18b, if it is not already connected. Once connected, the user, or the system, can initiate the transfer of fluid from source product bag 22 to the satellite product bag 36, resulting in a split fluid between product bag 22 and 36, as shown in FIG. 5. Each bag 22, 36 will contain an acceptable count of a component (platelets in the designated range) and an acceptable amount of fluid (fluid in the designated volume range).

Afterward each bag can undergo the PRT testing and treatment before use in a procedure or treatment.

The manner in which fluid is conveyed from one container to the other container may vary without departing from the scope of the present disclosure. In the illustrated embodiment, the middle support 14 is positioned at a greater elevation than the side supports, which allows for fluid flow from the source container to the satellite container or from the additive solution container to the source container via gravity. According to a gravity-based approach, the clamps are opened by the controller 21 to allow fluid to flow from one container or bag to another container or bag under the force of gravity while the controller 21 monitors the weights reported by the weight scales. Once the controller 21 determines that the proper amounts of fluid are in each container the controller actuates the clamps to prevent further flow from one container to another via the conduit.

In another embodiment, the system 10 may include a pump system configured to convey fluid from one container to the other container. The pump system (if provided) may be variously configured without departing from the scope of the present disclosure. In an exemplary embodiment in which the conduit is configured as a flexible tube, the pump system may include a peristaltic pump, for example, to convey fluid through the conduit.

Regardless of how fluid is conveyed from one container to the other container, once the proper amounts of fluid are in each container, the controller 21 may actuate a sealing system (if provided). The sealing system seals the conduit in at least one location to prevent fluid flow through the conduit, thereby ensuring that the proper amounts of fluid remain in each container at the end of a procedure. The sealing system may be configured to seal the conduit via a heat seal. The sealing system may be further configured to sever the conduit at the location(s) of the seal(s) to allow for separate transport, storage, and/or use of the containers. A seal may be severed by blade or the like or by any suitable approach, which may vary depending on the natures of the conduit and the seal.

It should be understood that the principles described herein are not limited to separation of a source product fluid into two parts, but rather a fluid may be separated into more than two parts and the principles described herein with regard to the system may be employed in providing a system for splitting a fluid into any number of parts. Although only shown with one satellite container in FIGS. 4 and 5, two or more satellite containers may be used to split an initial fluid product from a source container. The number of containers should also be noted in the process/procedure instructions provided by the used in the input. An exemplary fluid splitting procedure that can be done by the system is found in US Patent Publication No. 2022/0260408, which is incorporated by reference in its entirety.

If multiple satellite containers are utilized, fluid flow from the source container to the satellite containers may be initiated simultaneously or sequentially. Additionally, the controller 21 may be configured to allow for simultaneous flow into the satellite containers or may control the clamps (and the pump system, if provided) to allow for flow from the source container into only one satellite container at a time. This may include flowing fluid from the source container into one of the satellite containers until that satellite container is filled to the desired level before any fluid is conveyed from the source container into the other satellite container or may instead involve fluid being alternately conveyed into one satellite container and then the other, with the destination of fluid flow being changed multiple times before either satellite container is filled to the desired level.

The system 10 is shown as a standalone device, but it should be understood that they may be incorporated into a larger assembly or otherwise paired with another fluid processing device. For example, in one embodiment, if the fluid to be split is a biological fluid, such as a blood component (which may be, without limitation, a platelet product), the system may be paired with an apheresis system, such as the AMICUS® system manufactured by Fenwal, Inc. of Lake Zurich, Illinois, which is an affiliate of Fresenius Kabi AG of Bad Homburg, Germany. In such an implementation, blood is separated by the apheresis system into two or more components, with a platelet product (or other fluid) being produced by the apheresis system. The platelet product or other fluid is conveyed from the apheresis system directly into a source container being supported by the source support. With the platelet product or other fluid in the source container, a fluid splitting procedure of the type described above may be executed.

Incorporating the system 10 into a larger assembly or otherwise pairing it with another fluid processing device may include fluidly connecting a fluid flow circuit configured to be mounted to the system with a fluid flow circuit configured to be mounted to a separate fluid processing device. This may be achieved according to any suitable approach (e.g., using a luer connector), but in one embodiment, may be achieved by sterile connection of conduits of the two fluid flow circuits according to the approaches described in U.S. Pat. No. 9,199,070 or U.S. Pat. No. 10,040,247, both of which are hereby incorporated herein by reference.

Providing a fluid splitting system which can effectively split an initial fluid into fluid products with defined characteristics is extremely advantageous. By defining particular characteristics, such as component count range and volume range, it is ensured that a resulting fluid product can be utilized in the desired treatment or procedure.

### Aspects

Aspect 1. A method of splitting a fluid into fluid products with a component count within a designated component count range and volume within a designated volume range, comprising: (a) establishing a designated component count range and designated volume range for a fluid in each of a source container and at least one satellite container; (b) weighing a source container to determine the volume of fluid; (c inputting an initial component concentration and initial volume into the splitting device; (d) calculating the final component count and final volume for fluid in each of the source and at least one satellite container; (e) comparing the final component count to the designated component count range and the final volume to the designated volume range for each container; (f) calculating the amount of component additive solution to add the source container so that each of the source and at least one satellite container have a fluid with a final component count and final volume within the designated component count range and volume range; (g) flowing the amount of component additive solution from a component additive solution container into the source container; (h) repeating steps b-g until there is no amount needed at step (f) and the calculated final component count for each of the fluid in the source and at least one satellite container is within the designated component count range and the final volume is within the designated volume range; (i) flowing fluid from the source container of a fluid flow circuit to at least one satellite container of the fluid flow circuit by selectively allowing and preventing fluid flow from the source container to the at least one satellite container resulting in a satellite container and source container with a final component count within a designated component count range and a final volume within a designated volume range.

Aspect 2. The method of any of the preceding Aspects, wherein the fluid is a platelet product, the component is platelet, and the component additive solution is platelet additive solution.

Aspect 3. The method of any of the preceding Aspects, wherein the source container is positioned at a greater elevation than the at least one satellite container.

Aspect 4. The method of any of the preceding claims, further comprising measuring a component concentration.

Aspect 5. The method of any of the preceding Aspects, wherein the designated component count range is from 2.9×10¹¹ and 8.0×10¹¹.

Aspect 6. The method of any of the preceding Aspects, wherein the designated range for volume may be between 255 mL and 420 mL.

Aspect 7. The method of any of the preceding Aspects, wherein the designated component count range is from 2.9×10¹¹ and 8.0×10¹¹ and the designated range for volume is between 255 mL and 420 mL.

Aspect 8. The method of any of the preceding Aspects, wherein the fluid is flowed from the source container to the at least one satellite container via gravity.

Aspect 9. The method of any of the preceding Aspects, wherein the fluid is pumped from the source container to the at least one satellite container.

Aspect 10. The method of any one of the preceding Aspects, wherein the fluid flow circuit includes a plurality of satellite containers each fluidly connected to the source container.

Aspect 11. The method of any of the preceding Aspects, wherein establishing a designated component count range includes selecting a particular treatment procedure and setting the designated range based on procedure requirements.

Aspect 12. The method of any of the preceding aspects, wherein establishing a designated volume range includes selecting a particular treatment procedure and setting the designated range based on procedure requirements.

Aspect 13. A system for splitting a fluid, comprising: a source support configured to support a source container of a fluid flow circuit; at least one satellite support configured to support at least one satellite container of the fluid flow circuit fluidly connected to the source container; a weight scale associated with each of the supports; at least one clamp; an input; and a controller configured to: (a) establish a designated component count range and designated volume range for fluid in each of a source container and at least one satellite container; (b) weigh a source container to determine the volume of fluid; (c) input an initial component concentration and initial volume into the splitting device; (d) calculate the final component count and final volume for fluid in each of the source and at least one satellite container; (e) compare the final component count to the designated component count range and the final volume to the designated volume range for each container; (f) calculate the amount of component additive solution to add the source container so that each of the source and at least one satellite container have a fluid with a final component count and final volume within the designated component count range and volume range; (g) flow the amount of component additive solution from a component additive solution container into the source container; (h) repeat steps b-g until there is no amount needed at step (f) and the calculated final component count for each of the fluid in the source and at least one satellite container is within the designated component count range and the final volume is within the designated volume range; (i) flow fluid from the source container of a fluid flow circuit to at least one satellite container of the fluid flow circuit by selectively allowing and preventing fluid flow from the source container to the at least one satellite container resulting in a satellite container and source container with a final component count within a designated component count range and a final volume within a designated volume range.

Aspect 14. The system of Aspect 13, comprising a plurality of satellite supports, each configured to support a different satellite container fluidly connected to the source container and each including an associated weight scale.

Aspect 15. The system of any one of Aspects 13-14, configured for fluid flow from the source container to the satellite container via gravity.

Aspect 16. The system of any one of Aspects 13-15, further comprising a pump system, wherein the controller is configured to control the pump system to convey fluid from the source container to the satellite container.

Aspect 17. The system of any of Aspects 13-16, wherein the fluid is a platelet product, the component is platelet, and the component additive solution is platelet additive solution.

Aspect 18. The system of any of Aspects 13-17, wherein the source container is positioned at a greater elevation than the at least one satellite container.

Aspect 19. The system of any of Aspects 13-18, wherein the designated component count range is from 2.9×10¹¹ and 8.0×10¹¹.

Aspect 20. The system of any of Aspects 13-19, wherein the designated range for volume may be between 255 mL and 420 mL.

Aspect 21. The system of any of Aspects 13-20, wherein the designated component count range is from 2.9×10¹¹ and 8.0×10¹¹ and the designated volume range is between 255 mL and 420 mL.

Aspect 22. The system of any of Aspects 13-21, wherein establishing a designated component count range includes selecting a particular treatment procedure and setting the designated range based on procedure requirements.

Aspect 23. The system of any of Aspects 13-22, wherein establishing a designated volume range includes selecting a particular treatment procedure and setting the designated range based on procedure requirements.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

1. A method of splitting a fluid into fluid products with a component count within a designated component count range and volume within a designated volume range, comprising:
(a) establishing a designated component concentration range and designated volume range for a fluid in each of a source container and at least one satellite container;
(b) weighing a source container to determine the volume of fluid;
(c) inputting an initial component concentration and initial volume into the splitting device;
(d) calculating the final component count and final volume for fluid in each of the source and at least one satellite container;
(e) comparing the final component count to the designated component count range and the final volume to the designated volume range for each container;
(f) calculating the amount of component additive solution to add the source container so that each of the source and at least one satellite container has a fluid with a final component count and final volume within the designated component count range and volume range;
(g) flowing the amount of component additive solution from a component additive solution container into the source container;
(h) repeating steps b-g until there is no amount needed at step (f) and the calculated final component count for each of the fluid in the source and at least one satellite container is within the designated component count range and the final volume is within the designated volume range;
(i) flowing fluid from the source container of a fluid flow circuit to at least one satellite container of the fluid flow circuit by selectively allowing and preventing fluid flow from the source container to the at least one satellite container resulting in a satellite container and source container with a final component count within a designated component count range and a final volume within a designated volume range.

2. The method of claim 1, wherein the fluid is a platelet product, the component is platelet, and the component additive solution is platelet additive solution.

3. The method of claim 1, wherein the source container is positioned at a greater elevation than the at least one satellite container.

4. The method of claim 1, further comprising measuring a component concentration.

5. The method of claim 1, wherein the designated component count range is from 2.9×10¹¹ and 8.0×10¹¹.

6. The method of claim 1, wherein the designated range for volume may be between 255 mL and 420 mL.

7. The method of claim 1, wherein the designated component count range is from 2.9×10¹¹ and 8.0×10¹¹ and the designated range for volume is between 255 mL and 420 mL.

8. The method of claim 1, wherein the fluid is flowed from the source container to the at least one satellite container via gravity.

9. The method of claim 1, wherein the fluid is pumped from the source container to the at least one satellite container.

10. The method of claim 1, wherein the fluid flow circuit includes a plurality of satellite containers each fluidly connected to the source container.

11. The method of claim 1, wherein establishing a designated component count range includes selecting a particular treatment procedure and setting the designated range based on procedure requirements.

12. The method of claim 1, wherein establishing a designated volume range includes selecting a particular treatment procedure and setting the designated range based on procedure requirements.

13. A system for splitting a fluid, comprising:
a source support configured to support a source container of a fluid flow circuit;
at least one satellite support configured to support at least one satellite container of the fluid flow circuit fluidly connected to the source container;
a weight scale associated with each of the supports;
at least one clamp;
an input; and
a controller configured to
(a) establish a designated component count range and designated volume range for fluid in each of a source container and at least one satellite container;
(b) weigh a source container to determine the volume of fluid;
(c) input an initial component concentration and initial volume into the splitting device;
(d) calculate the final component count and final volume for fluid in each of the source and at least one satellite container;
(e) compare the final component count to the designated component count range and the final volume to the designated volume range for each container;
(f) calculate the amount of component additive solution to add the source container so that each of the source and at least one satellite container has a fluid with a final component count and final volume within the designated component count range and volume range;
(g) flow the amount of component additive solution from a component additive solution container into the source container;
(h) repeat steps b-g until there is no amount needed at step (f) and the calculated final component count for each of the fluid in the source and at least one satellite container is within the designated component count range and the final volume is within the designated volume range;
(i) flow fluid from the source container of a fluid flow circuit to at least one satellite container of the fluid flow circuit by selectively allowing and preventing fluid flow from the source container to the at least one satellite container resulting in a satellite container and source container with a final component count within a designated component count range and a final volume within a designated volume range.

14. The system of claim 13, comprising a plurality of satellite supports, each configured to support a different satellite container fluidly connected to the source container and each including an associated weight scale.

15. The system of claim 13, configured for fluid flow from the source container to the satellite container via gravity.

16. The system of claim 13, further comprising a pump system, wherein the controller is configured to control the pump system to convey fluid from the source container to the satellite container.

17. The system of claim 13, wherein the fluid is a platelet product, the component is platelet, and the component additive solution is platelet additive solution.

18. The system of claim 13, wherein the source container is positioned at a greater elevation than the at least one satellite container.

19. The system of claim 13, wherein the designated component count range is from 2.9×10¹¹ and 8.0×10¹¹.

20. The system of claim 13, wherein the designated range for volume may be between 255 mL and 420 mL.

21. The system of claim 13, wherein the designated component count range is from 2.9×10¹¹ and 8.0×10¹¹ and the designated range for volume is between 255 mL and 420 mL.

22. The system of claim 13, wherein establishing a designated component count range includes selecting a particular treatment procedure and setting the designated range based on procedure requirements.

23. The system of claim 13, wherein establishing a designated volume range includes selecting a particular treatment procedure and setting the designated range based on procedure requirements.
